(19) United States
(12) Patent Application Publication
(10) Pub. No.: US 2011/0319829 A1
(43) Pub. Date: Dec. 29, 2011

(54) Patient-Wearable System, Twist Isolating Device, And Method

(76) Inventors: Richard Livesay, Peoria, IL (US); Teresa Livesay, Peoria, IL (US); Kevin Livesay, Long Grove, IL (US)

(21) Appl. No.: 13/170,658

(22) Filed: Jun. 28, 2011

Related U.S. Application Data
(60) Provisional application No. 61/359,555, filed on Jun. 29, 2010.

(51) Int. Cl. A61M 25/02

(52) U.S. Cl. 604/179

(57) ABSTRACT

A patient-wearable system includes a drainage/supply mechanism having a proximal and distal tube, and a twist isolation mechanism defining a twisting force diffusion path for limiting transmitting twisting force between the proximal and distal tubes during passing fluids between an intraluminal device and a fluid reservoir. The twist isolation mechanism includes a mounting component, a control component, and a conduit component, the control component including first and second load reacting outboard body edges and the conduit component defining a middle lumen configured to fluidly connect with each of the proximal and distal tubes via proximal and distal couplings, and being mounted to the control component.

### CROSS-REFERENCE TO RELATED PATENT APPLICATIONS

This application claims priority to provisional U.S. Patent Application Ser. No. 61/359,555, filed Jun. 29, 2010, entitled “Patient-Wearable System, Twist Isolating Device, And Method.”

### TECHNICAL FIELD

The present disclosure relates generally to systems and strategies for draining or supplying fluids from or to a patient, and relates more particularly to isolating twisting of a proximal tube from a distal tube in a patient-wearable system.

### BACKGROUND

All manner of medical devices, systems and instrumentation are routinely secured at least in part to a patient's body. Electronic monitoring devices, IV drip tubes, and drainage mechanisms are notable examples. The reasons for securing such devices or parts of such devices and systems to a patient's limb or torso, for example, include convenience as well as patient mobility. The advantages of enabling a patient to walk around a hospital, clinic, or even their home, without needing to be tethered to medical equipment, are well recognized.

A classic, if relatively unsophisticated, technique for securing tubes and the like to a patient's body is the simple use of medical tape. Those skilled in the art will be familiar with the use of medical tape to attach IV tubes to a patient's arm or leg, for example. While such an approach has the obvious advantages of simplicity, rapid application, and low cost, few have never experienced the discomfort of removing medical tape from one's skin. In addition to the obvious downsides of subjecting a patient to pain, the adhesive qualities of tape tend to degrade over time, particularly in response to environmental moisture such as sweating.

A wide variety of wearable holsters, harnesses, wristbands and the like have been developed over the years in an attempt to provide a more sophisticated, and in some cases more technically specialized, approach to securing medical devices, systems, and instrumentation to human patients. One example is known from the field of urinary collection systems, in which a wearable collection bag and fluid routing system is provided, to enable draining of urine from a catheter placed within the patient's body. The collection bag is typically strapped to a patient's leg, a catheter placed appropriately, and a tube routed between the bag and catheter. It is common for the tube to be taped or strapped to the patient between the catheter and collection bag. Such systems have seen some commercial success, however, they are not without disadvantages.

When a patient moves his or her body, the relative positioning in space of different parts of a wearable system tends to change. Components such as tubes may be tensioned or compressed, buckling systems loosened, and the components twisted relative to one another. In the case of the well known taping approach, pulling on medical tape adhered to a patient's skin can hurt, cause the tape to detach, shift or slip, or otherwise hasten the end of the tape's service period, requiring intervention by medical personnel or the patient himself. With many known wearable type systems, movement of the patient's body can cause momentary changes in strap tension, causing parts of the system to slip or even decouple from the patient's body completely. Compounding the problems with conventional approaches is the fact that most systems are worn under a patient's clothing or hospital gown, making access to adjust, re-tape, or reconnect components at best annoying, and in many cases difficult for health impaired individuals.

### SUMMARY OF THE DISCLOSURE

In one aspect, a patient-wearable system for draining or supplying fluids includes a drainage/supply mechanism having a proximal tube and a distal tube, the proximal tube defining a fluid inlet and a proximal lumen fluidly connecting with the fluid inlet, and the distal tube defining a fluid outlet and a distal lumen fluidly connecting with the fluid outlet. The system further includes a twist isolation mechanism having a mounting component, a control component, and a conduit component. The mounting component includes a flexible retention strap coupled with the control component and configured to form an adjustable loop about a limb or torso of a patient. The control component includes a rigid body having a proximal body edge, a distal body edge, and first and second load reacting outboard body edges. The conduit component is mounted to the control component and defines a middle lumen. The system further includes a proximal coupling positioned between the proximal tube and the conduit component, and a distal coupling positioned between the distal tube and the conduit component. Each of the proximal and distal couplings have a release state at which the corresponding proximal tube or distal tube is rotatable relative to the conduit component, and a locking state. The system further defines a fluid flow path which includes the proximal, distal, and middle lumens, for passing fluids between an intraluminal device and a fluid reservoir. The twist isolation mechanism defines a twisting force diffusion path between the proximal and distal couplings and the control component, for limiting transmitting twisting force between the proximal tube and the distal tube during passing fluids via the fluid flow path.

In another aspect, a method of passing fluids between a patient and a fluid reservoir includes establishing a fluid flow path between and intraluminal device and a fluid reservoir via a fluid drainage/supply mechanism at least in part by fluidly connecting a proximal tube of the fluid supply/drainage mechanism with the intraluminal device, and fluidly connecting a distal tube of the fluid drainage/supply mechanism with the fluid reservoir. The method further includes establishing a twisting force diffusion path between the fluid drainage/supply mechanism and a limb or torso of the patient at least in part by locking the proximal tube to a conduit component of a twist isolation mechanism, locking the distal tube to the conduit component of the twist isolation mechanism, and securing the twist isolation mechanism to the limb or torso of the patient. The method further includes passing fluid between the intraluminal device and the fluid reservoir via a fluid lumen defined by the proximal tube, the distal tube, and the conduit component. The method further includes isolating twisting of the proximal tube from twisting of the distal tube during passing the fluid, at least in part by transferring a twisting force according to the twisting force diffusion path from the proximal tube or the distal tube to the limb or torso of the patient.

In still another aspect, a patient-wearable device for isolating twisting of a proximal tube from a distal tube and a fluid drainage/supply system includes a mounting component having a flexible strap with first and second strap ends, and being configured to form an adjustable loop about a limb or torso of a patient. The device further includes a control component coupled with the first and second strap ends, and including a rigid body having a proximal body edge, a distal body edge, and first and second load reacting outboard body edges. The device further includes a conduit component mounted to the control component and defining a fluid lumen having a proximal lumen end and a distal lumen end. The device further includes a proximal coupling component and a distal coupling component connected with the conduit component, and each being configured to lockingly connect with a tube of the fluid drainage/supply system. The control component further includes a patient contact surface having a uniform profile extending from the first outboard body edge to the second outboard body edge, and an upper surface opposite the patient contact surface having a non-uniform profile extending from the first outboard body edge to the second outboard body edge. The non-uniform profile includes a raised profile segment defined by the conduit component, and first and second low profile segments defined by the control component. The fluid lumen includes a segment of a fluid flow path for passing fluids between an intraluminal device and a fluid reservoir, and the patient-wearable device defines a twisting force diffusion path between the conduit component and the control component, for limiting transmitting twisting force between the proximal coupling and the distal coupling during passing fluids between the intraluminal device and the fluid reservoir.

### BRIEF DESCRIPTION OF THE DRAWINGS

FIG. 1 is a pictorial view of a system, in service on a patient, according to one embodiment;

FIG. 2 is a fragmentary view of a portion of the system of FIG. 1;

FIG. 3 is a perspective view of a portion of the system of FIG. 1;

FIG. 4 is a top view of a portion of the system shown in FIGS. 1-3;

FIG. 5 is a partial, sectioned side diagrammatic view of a device according to one embodiment;

FIG. 6 is a partial, sectioned side diagrammatic view of a device, according to another embodiment;

FIG. 7 is a top view of the device of FIG. 6;

FIG. 8a is a different pictorial view of the system of FIG. 1;

FIG. 8b is yet another view of the system of FIG. 8b; and

FIG. 9 is a top view of a portion of a system, according to yet another embodiment.

### DETAILED DESCRIPTION

The present disclosure provides systems and methods relating to draining or supplying fluids from or to a patient. The following description, in conjunction with the attached drawings, discloses a number of practical implementation strategies.

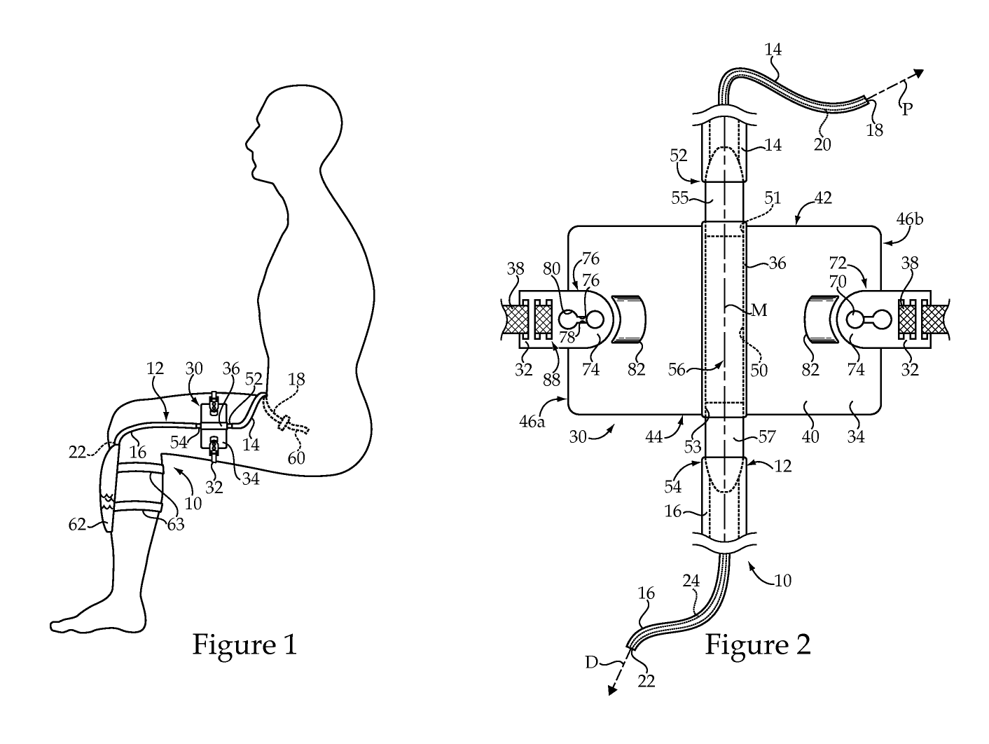

Referring to FIG. 1, there is shown a patient-wearable system 10 for draining or supplying fluids between a patient and a fluid reservoir. Those skilled in the art will appreciate that certain medical devices, systems and methods for conveying fluid between a patient and a fluid reservoir utilize similar or identical hardware regardless of whether used for draining body fluids or for supplying treatment fluids to the body. Accordingly, it should be appreciated that the present disclosure includes teachings that may be advantageously applied to draining body fluids, such as urine, lymph, and blood, as well as to supplying treatment fluids to the human body such as saline, analgesics, thrombolytic agents, chemotherapeutic agents, diagnostic agents, and still others.

System 10 may include a drainage/supply mechanism 12 having a proximal tube 14, and a distal tube 16. Proximal tube 14 may be configured to fluidly connect with an intraluminal device 60, whereas distal tube 14 may be configured to connect with a fluid reservoir 62. In one embodiment, device 60 may include an intraluminal drainage device such as a urinary catheter, and reservoir 62 may include a fluid collection device such as a leg bag for containing urine or another body fluid drained from device 60 by way of mechanism 12. Reservoir 62 might include a soft textile or foam coating, potentially attached via a hook and loop mechanism such as Velcro®, to enhance patient comfort. In other embodiments, device 60 might include an infusion catheter, an intravenous needle, or still another device configured to deliver treatment fluid into a body lumen, and reservoir 62 might include an extraluminal fluid supply reservoir such as an IV bag.

System 10 may also include a twist isolation mechanism 30 having a mounting component 32, a control component 34 and a conduit component 36. Mounting component 32 may include a flexible retention strap 38 of a textile material or the like, coupled with control component 34 and configured to form an adjustable loop about a limb or torso of a patient. In the illustrated embodiment, twist isolation mechanism 30 is mounted to a patient's thigh, and reservoir 62 is mounted to a patient's calf by way of a set of straps 63.

Referring also to FIG. 2, proximal tube 14 may define a fluid inlet 18 and a proximal lumen 20 fluidly connecting with fluid inlet 18. Distal tube 16 may define a fluid outlet 22 and a distal lumen 24 fluidly connecting with fluid outlet 22. A proximal coupling 52 is positioned between proximal tube 14 and conduit component 36, and a distal coupling 54 is positioned between distal tube 16 and conduit component 36. Proximal coupling 52 may include a coupling component 55, and distal coupling 54 may include another coupling component 57. Each of proximal and distal couplings 52 and 54 may include a release state at which the corresponding proximal or distal tube 14 or 16 is rotatable relative to conduit component 36, and a locking state. In one embodiment, each of tubes 14 and 16 may be formed from a plastically deformable material such as nylon. Each of coupling components 55 and 57 may include a tapered bullet fitting positioned within conduit component 36, and having a narrowing taper in a direction toward tubes 14 and 16, respectively, such that tube 14 and tube 16 may be slid onto components 55 and 57 by deforming the engaged ends of tubes 14 and 16 and locking tubes 14 and 16 against rotation. Accordingly, the locking state of each of couplings 52 and 54 may be understood as a friction locking state, although alternatives are contemplated such as a clamping mechanism, threads or the like.

Each of control component 34 and conduit component 36 may be formed from a material such as nylon, inherently including some degree of deformability, but also being relatively more rigid than flexible strap 38. To this end, control component 34 may include a rigid body 40. Body 40 may include a proximal body edge 42, a distal body edge 44, and first and second load reacting outboard body edges 46a and 46b, the significance of which will be apparent from the following description. Conduit component 36 may be mounted to control component 34 and defines a middle lumen 50 having a proximal lumen end 51 and a distal lumen end 53. In one embodiment, conduit component 36 and control component 34 may be formed as a one-piece molded body, such as by way of an extrusion process. In further embodiments, a relatively long extrusion could be cut into multiple segments each including a control component and conduit component similar to that described herein. System 10 may also define a fluid flow path 56 which includes proximal lumen 20, distal lumen 24, and middle lumen 50, for passing fluids between device 60 and reservoir 62. Middle lumen 50 may be unobstructed from one axial terminal end of conduit component 36 to the other, such that fluid always flows freely between lumens 20 and 24. Each of lumens 20, 24, 50 may thus be understood to include a segment of flow path 56. Twist isolation mechanism 30 may also define a twisting force diffusion path between proximal and distal couplings 52, for limiting transmitting twisting force between proximal tube 14 and distal tube 16 during passing fluids via fluid flow path 56, as further described herein.

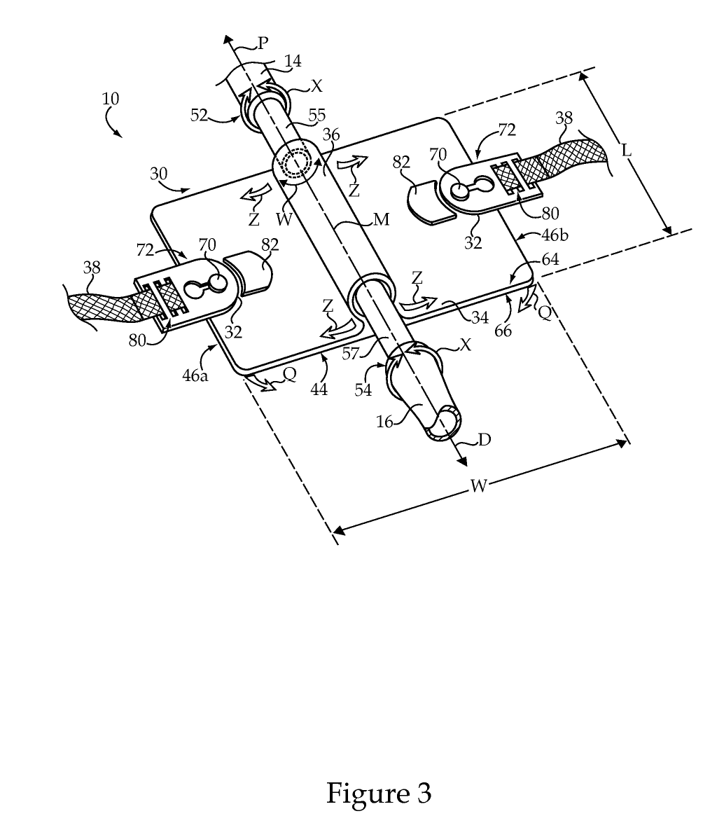

Referring now also to FIG. 3, control component 34 may include an upper surface 64, and a lower surface 66. Lower surface 66 may define a fixed length segment of the adjustable loop formed by flexible retention strap 38. In the embodiment shown, mechanism 30 includes a total of one conduit component 36 which is positioned equidistant first and second outboard body edges 46a and 46b. In other embodiments, more than one conduit component might be provided, for example where multiple drainage or supply pathways are used. It may further be noted that conduit component 36 comprises an enclosed tubular configuration; however, in other embodiments conduit component 36 might include a partially cylindrical component having an opening adapted to receive a separate tube component which defines lumen 50 therein, and deformable to retain such separate tube by way of a snap-fit or the like. As also shown in FIG. 3, control component may include a length dimension L extending from proximal body edge 42 to distal body edge 44, and a width dimension W extending from first outboard body edge 46a to second outboard body edge 46b. Width dimension W may be greater than length dimension L in one embodiment. It may still further be noted from FIG. 3 that lower surface 66 includes a uniform profile extending from first outboard body edge 46a to second outboard body edge 46b. Lower surface 66 may include a patient contact surface of twist isolation mechanism 30 configured to contact the limb or torso of a patient to which mechanism 30 is secured. The uniform profile of lower surface 66 may include a planar profile, or a modestly curving profile enabling surface 66 to readily conform to patient anatomy. A comfort material such as a pad or the like (not shown) might be affixed to lower surface 44 in one embodiment to provide a padded and/or humidity resistant or even hypoallergenic or antibacterial contact interface for contacting a patient's skin. The pad might include a textile or an open cell foam material, for instance.

Upper surface 64 may include an exposed upper surface of mechanism 30, having a non-uniform profile extending from first outboard body edge 46a to second outboard body edge 46b. The profile of upper surface 64 may be understood to be non-uniform relative to lower surface 66. The non-uniform profile may include a raised profile segment between outboard body edges 46a and 46b, defined by conduit component 36, and first and second low profile segments between conduit component 36 and each of outboard body edges 46a and 46b, defined by control component 34. In the illustrated embodiment, a set of buckling posts 70 are located upon and stand proud of upper surface 64. Buckling posts 70 may be formed integrally during molding control component 34 and conduit component 36, for example, but could also include separate parts attached to control component 34 via an adhesive, for example. Buckling posts 70 are shown coupled with mounting component 32 to enable mechanism 30 to be secured to a patient's limb or torso via mounting component 38. Additional details of buckling posts 70 and mounting component 32 are further described below.

As alluded to above, twist isolation mechanism 30 may be used to limit transmitting twisting forces between proximal tube 14 and distal tube 16 during passing fluids via fluid flow path 56. In this vein, proximal lumen 20 may define a first longitudinal axis P having a mutable configuration, and distal lumen 24 may define a second longitudinal axis D also having a mutable configuration. Axes P and D may be understood as having mutable configurations relative to a middle longitudinal axis M defined by middle lumen 50. Axis M may include a fixed, linear configuration. In other words, tubes 14 and 16 may be permitted, and expected, to move relative to twist isolation mechanism 30 and relative to the patient's body as the patient moves. As a result, the paths through space traversed by axes P and D may change relative to the patient's body, and in response to movement of the patient's body. The path through space traversed by axis M, however, will tend not to change relative to the patient's body in response to his or her movement. This property associated with axis M, and other properties of twist isolation mechanism 30, can enable twisting forces on one or both of tubes 14 and 16 to be isolated from one another to improve patient comfort and maintain various components of system 10 mounted upon the patient's body in the manner intended, instead of decoupling or sliding to areas at which discomfort to the patient or problems in operation of system 10 can result.

The twisting force diffusion path defined by mechanism 30 may include a compound twisting force diffusion path having a plurality of different path segments. The twisting force diffusion paths contemplated herein result at least in part from the selected geometry of certain the components of system 10. In general, it may be noted that each of tubes 14 and 16 includes a relatively wider outer diameter dimension than coupling components 55 and 57. Each of coupling components 55 and 57 includes a relatively narrower outer diameter dimension than conduit component 36. Those skilled in the art will appreciate that transfer of a twisting force or torque from one component of a certain size or material thickness to another component of a different size or material thickness may be associated with differences in relative stress and/or strain on the material of the respective components. By way of analogy, applying a torque to a fastener such as a screw to drive the screw into another component will tend to induce a stress and strain on material of the screw. The component into which the screw is driven will tend to experience a stress or strain in response to the applied torque. The stress and strain on the receiving component will, at least in most familiar examples, be diffused into a relatively larger volume of material. The reverse also tends to be true where a larger or thicker component is torqued relative to a smaller or thinner component.

In a generally analogous manner, transmitting a torque from tube 14 or 16 to the connected coupling component 55 or 57 will tend to gather or concentrate the torque in passing from the larger diameter tube 14 or 16 to the smaller diameter coupling component 55 or 57. Transmitting the torque from coupling component 55 or 57 to conduit component 36 will tend to diffuse the torque, and transmitting the torque from conduit component 36 to control component 34 will tend to further diffuse the torque. It has been discovered that the configuration of components described herein in which twisting forces are gathered or concentrated, then diffused, then further diffused, provides a practical implementation strategy for limiting transmitted twisting forces between tubes 14 and 16, as further described herein.

In one embodiment, the twisting force diffusion path may include force gathering path segments between each of tubes 14 and 16 and coupling components 55 and 57. The force gathering path segments are shown in FIG. 3 via arrows X, and may include radial force gathering path segments. From coupling components 55 and 57 to conduit component 36, the twisting force diffusion path may include radial force distributing path segments, shown via arrows W. From conduit component 36 to control component 34, the twisting force diffusion path may include tangential force distributing path segments, shown via arrows Z. The path segments illustrated via arrows Z may be understood to be tangential to a cylinder centered on axis M and defined by conduit component 36. The terms “radial” and “tangential” are used herein in a relative sense, as the paths traversed by forces transmitted through mechanism 30 will generally not be purely radial or tangential, but nevertheless generally so. Arrows Q represent an approximate path followed by forces transmitted from load reacting outboard body edges to a limb or torso of a patient.

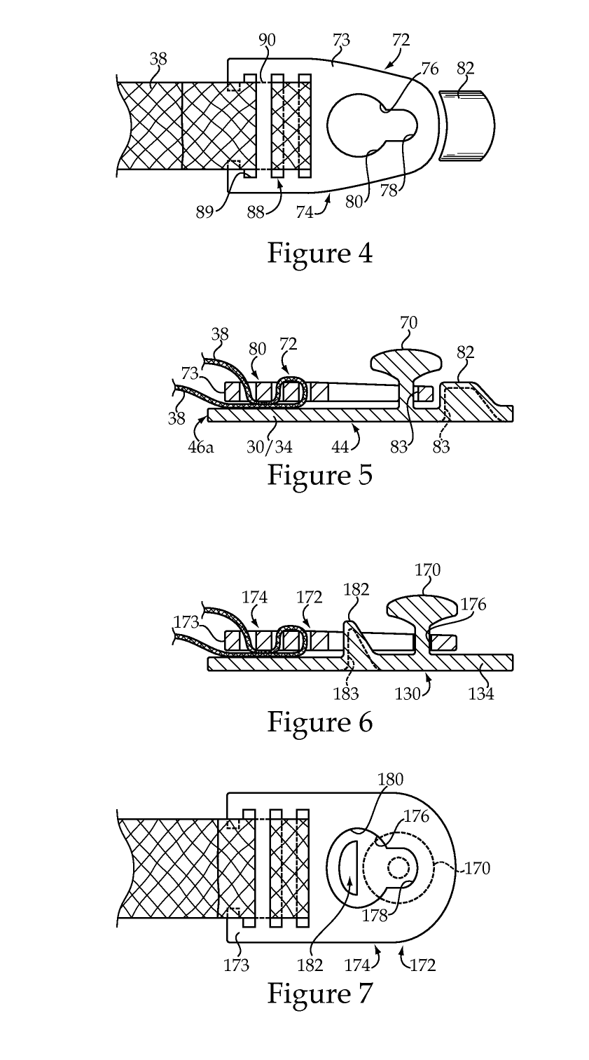

Referring now also to FIGS. 4 and 5, there is shown additional features of buckling mechanism 72. Buckling mechanism 72 may include a buckle 74 which includes a body 73 attached to strap 38. Body 73 may define a compound aperture 76 which is positioned about buckling post 70. Buckle 74 may include an advanced position at which buckling post 70 is positioned within a major opening 80 of compound aperture 76, and a locked position at which post 70 is positioned within a minor opening 78 of aperture 76. In FIG. 2, buckle 74 is shown at the locked position such that post 70 is positioned within minor opening 78. Minor opening 78 may be sized such that decoupling of buckle 74 is inhibited by interference with the relatively larger sized post 70. At the advanced position, where buckling post 70 is positioned within major opening 80, buckle 74 may be decoupled from post 70.

In the illustrated embodiment, mounting component 32 includes two buckling mechanisms each identified with reference numeral 72. In other embodiments contemplated herein, only one buckling mechanism 72 might be used, coupled with one end of strap 38, while the other end of strap 38 is attached to control component 34 via some other mechanism. Buckling mechanism 72 may also include a strap length customizing mechanism 88 which includes a plurality of ribs 90 alternating with a plurality of slots 89 such that strap 38 may be fed through slots 89, over and under ribs 90, to frictionally engage strap 38 at an adjustable engagement location with body 73. By changing the position of strap 38 between and among slots 89 and ribs 90, the engagement location of strap 38 with buckle 74 may be adjusted to change a loop length of strap 38 defined by customizing mechanism 88. Buckling mechanism 72 may further include a means for locking buckle 74 at the locked location at which post 70 is positioned within minor opening 78. In one embodiment, as shown in FIGS. 2-5, the means for maintaining buckle 74 at the locked position includes a button 82 which is formed in control component 34 and stands proud of upper surface 64. Button 82 may include a depressible button defining an air space 83, enabling button 82 to be pushed downward such that button 82 ceases to inhibit moving buckle 74 from its locked position toward its advanced position. In alternative embodiments, a separate means for locking might be omitted, as strap tension or frictional engagement between buckle 74 and post 70 may be sufficient to keep the respective components locked together when positioned about a patient.

Referring now to FIGS. 6 and 7, there is shown a buckling mechanism 172 according to another embodiment. Buckling mechanism 172 may be used with a control component 134 of a twist isolation mechanism 130 having similarities with twist isolation mechanism 30 discussed above, but also certain differences. Buckling mechanism 172 may include a buckle 174 having a body 173, and being movable between a locked position and an advanced position to position a post 170 within a major opening 180 or a minor opening 178 of a compound aperture 176 defined by body 173. In contrast to buckling mechanism 72, in the embodiment of FIGS. 6 and 7 a depressible button 182 of control component 134 is positionable within compound aperture 176. Button 182 may define an air pocket 183 and may be manually depressed such that body 173 may be advanced to decouple buckle 174 from post 170. When button 182 is not depressed, it will tend to extend upwardly into major opening 180 of compound aperture 176 to inhibit moving of buckle 174.

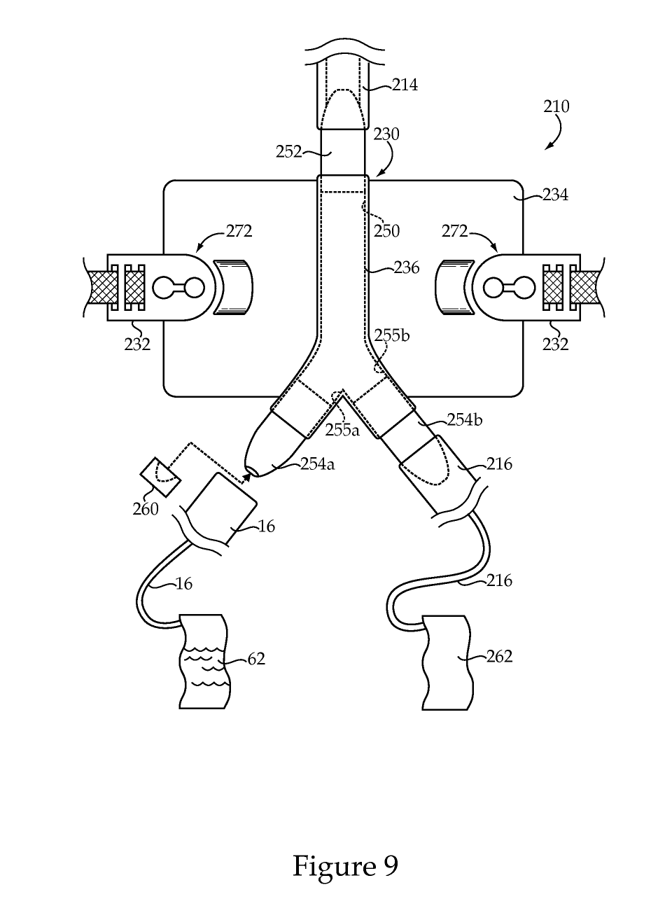

Turning now to FIG. 9, there is shown a portion of a patient-wearable system 210 for draining or supplying fluids according to another embodiment. System 210 has certain similarities with the embodiments described above, but also certain differences. System 210 may include a proximal tube 214, a proximal coupling having a coupling component 252, and a twist isolation mechanism 230 connected with proximal tube 214 by way of component 252. Twist isolation mechanism 230 may include a mounting component 232 including a buckling mechanism 272, for example. Twist isolation mechanism 230 may further include a control component 234 having a configuration similar to the configuration of control component 34 discussed above. Twist isolation mechanism 230 may also include a conduit component 236 defining a fluid lumen 250.

In contrast to the embodiment described above, conduit component 236 may define a first branch lumen 255a fluidly connecting with lumen 250, and a second branch lumen 255b fluidly connecting with fluid lumen 250. First and second branch lumens 255a and 255b may be arranged in a Y-shape. Twist isolation mechanism 230 may further include a first distal coupling component 254a and a second distal coupling component 254b. In FIG. 9, system 210 is shown as it might appear where distal tube 16, fluidly connecting with a full fluid reservoir 62, has just been disconnected from component 254a. Distal coupling component 254b is shown having been connected with a second distal tube 216 which connects with an empty fluid reservoir such as an empty fluid collection bag 262. A cap, plug or the like 260 is also shown in FIG. 9 and may be connected with distal coupling component 254a to provide a fluid seal when no fluid collection device is connected. The embodiment shown in FIG. 9 may be used to swap a full collection bag for an empty connection bag without needing to disconnect other parts of system 210 or decouple them from a patient's body. It will be readily appreciated that tube 16 and reservoir 62 may be disconnected from conduit component 236 after or simultaneous with connecting distal tube 216 and fluid reservoir 262, enabling a simple and efficient method for switching fluid collection bags or the like.

### INDUSTRIAL APPLICABILITY

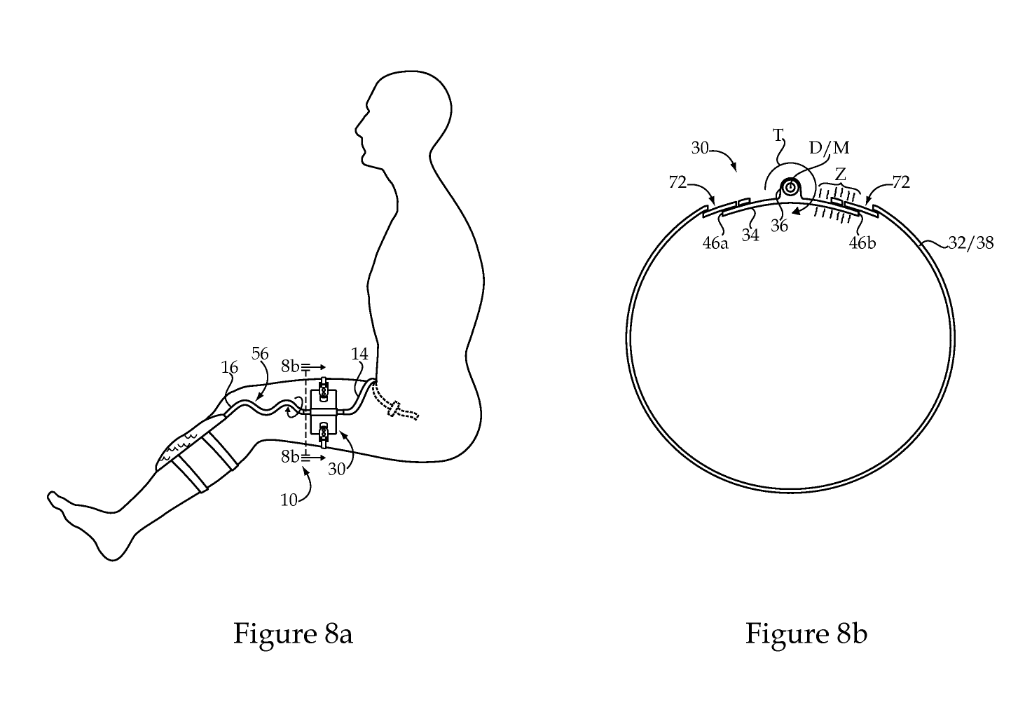

Referring to the Figures generally, and in particular to FIG. 8a, it will be recalled that fluid flow path 56 may be established between device 60 and fluid reservoir 62 by connecting proximal tube 16 with device 60, and fluidly connecting distal tube 14 with fluid reservoir 62. Performing these steps of connecting the various components may establish fluid flow path 56. Locking proximal tube 14 and locking distal tube 16 to conduit component 36, and securing twist isolation mechanism 30 to a limb or torso of a patient may establish a twisting force diffusion path between mechanism 12 and the limb or torso of the patient, as described herein. With system 10 assembled and secured to a patient, fluid may be passed between device 60 and reservoir 62. Where device 60 includes a drainage catheter or some other drainage mechanism, a body fluid may be drained via lumens 20, 50, and 24 to fluid reservoir 62.

As discussed above, when a patient moves his or her body, devices such as fluid drainage or supply devices may by necessity also move. In many instances, moving a patient's body or a part of the patient's body can result in certain medical device components experiencing twisting. In the FIG. 8a, system 10 is shown as it might appear where the patient has moved his or her body in such a way that a twisting force has been imparted to distal tube 16. It should be appreciated that the illustrated movement of the patient's body is but one example of many different movements which might result in imparting a twisting force to a part of the patient's body. Moreover, the twisting forces may include compound forces such that both a twisting force, and compressive or expansive forces on a part of system 10 such as tube 16 could result.

In any event, applying a twisting force to one of proximal tube 14 or distal tube 16, will typically not result in the undesired shifting, decoupling, discomfort or other consequences of patient movement associated with known systems. In particular, twist isolation mechanism 30 may isolate twisting of proximal tube 14 from twisting of distal tube 16 during passing fluid through system 10 at least in part by transferring the twisting force from the proximal tube or the distal tube to the limb or torso of the patient, rather than transferring the twisting force between tubes 14 and 16. Shifting or other undesired consequences which would result from transmitted the twisting force from tube 14 to tube 16 can be avoided.

In FIG. 8b, it may be noted that a twisting force, denoted via reference letter T is being applied to twist isolation mechanism, and in particular to conduit component 36, in a clockwise direction about longitudinal axis D/M. The twisting force T may be transmitted according to the twisting force diffusion path defined by mechanism 30 to control component 34, and thenceforth to the patient's limb or torso. A portion of the twisting force T, however, may be stored within twist isolation mechanism 30, in particular within control component 34. A region Z is identified in FIG. 8b which illustrates a portion of control component 34 which is placed in tension and/or compression, and stores said portion of the twisting force. It may be noted that region Z is adjacent outboard body edge 46b. When the patient moves their body again, the stored portion of the twisting force may be returned to the corresponding proximal or distal tube 14 and 16. It may be understood, however, that during returning the stored portion of the twisting force, the force may actually be concentrated rather than diffused. In the illustrated case, a twisting force applied to distal tube 16 is transmitted to a portion of control component 34 adjacent outboard body edge 46b and stored therein via the resilience of the material from which control component 34 is formed. When the patient moves their body again, the stored portion of the twisting force may be returned to distal tube 16.

As alluded to above, the present disclosure is contemplated to provide significant improvements over state of the art strategies for securing various medical devices, and in particular fluid supply and/or drainage mechanisms, to a patient's body. Rather than a mounting strategy with provides discomfort to the patient, or causes undesired events such as decoupling of bag retention mechanisms or other components from a patient's body, when a system according to the present disclosure experiences twisting forces, the forces may be smoothly transmitted to the patient's body, a portion of the force stored, and then returned back to the system when the patient again moves in a generally opposite manner.

The present description is for illustrative purposes only, and should not be construed to narrow the breadth of the present disclosure in any way. Thus, those skilled in the art will appreciate that various modifications might be made to the presently disclosed embodiments without departing from the full and fair scope and spirit of the present disclosure. Other aspects, features and advantages will be apparent upon an examination of the attached drawings and appended claims.

What is claimed is:

1. A patient-wearable system for draining or supplying fluids comprising:
a drainage/supply mechanism including a proximal tube and a distal tube, the proximal tube defining a fluid inlet and a proximal lumen fluidly connecting with the fluid inlet, and the distal tube defining a fluid outlet and a distal lumen fluidly connecting with the fluid outlet;
a twist isolation mechanism including a mounting component, a control component, and a conduit component, the mounting component including a flexible retention strap coupled with the control component and configured to form an adjustable loop about a limb or torso of a patient, the control component including a rigid body having a proximal body edge, a distal body edge, and first and second load reacting outboard body edges, and the conduit component being mounted to the control component and defining a middle lumen;
a proximal coupling positioned between the proximal tube and the conduit component, and a distal coupling positioned between the distal tube and the conduit component, each of the proximal and distal couplings having a release state at which the corresponding proximal tube or distal tube is rotatable relative to the conduit component, and a locking state; and
the system defining a fluid flow path which includes the proximal, distal, and middle lumens, for passing fluids between an intraluminal device and a fluid reservoir, and the twist isolation mechanism defining a twisting force diffusion path between the proximal and distal couplings and the control component, for limiting transmitting twisting force between the proximal tube and the distal tube during passing fluids via the fluid flow path.

2. The system of claim 1 wherein the proximal lumen defines a first longitudinal axis having a mutable configuration and the distal lumen defines a second longitudinal axis also having a mutable configuration, and wherein the middle lumen defines a middle longitudinal axis extending from the proximal coupling to the distal coupling and having a fixed, linear configuration.

3. The system of claim 2 wherein the control component includes an upper surface, a lower surface defining a fixed length segment of the adjustable loop, and a buckling post standing proud of the upper surface and coupled with the mounting component.

4. The system of claim 3 wherein:
the mounting component further includes a buckling mechanism having a buckle attached to the flexible strap, and defining a compound aperture positioned about the buckling post, the compound aperture including a minor opening and a major opening; and
the buckle having an advanced position at which the at least one buckling post is positioned within the major opening, and a locked position at which the buckling post is positioned within the minor opening.

5. The system of claim 2 wherein:
the control component includes a length dimension extending from the proximal body edge to the distal body edge, and a width dimension extending from the first outboard body edge to the second outboard body edge, the width dimension being greater than the length dimension;
the lower surface includes a patient contact surface of the twist isolation mechanism having a uniform profile extending from the first outboard body edge to the second outboard body edge; and
the upper surface includes an exposed upper surface of the twist isolation mechanism having a non-uniform profile extending from the first outboard body edge to the second outboard body edge, the non-uniform profile including a raised profile segment defined by the conduit component, and first and second low profile segments defined by the control component.

6. The system of claim 5 wherein each of the proximal coupling and the distal coupling includes a tapered bullet fitting, and wherein the locking state includes a friction locking state with the corresponding proximal or distal tube.

7. The system of claim 5 wherein the twist isolation mechanism includes a total of one conduit component, and the conduit component is positioned equidistant the first and second outboard body edges.

8. The system of claim 1 wherein the middle lumen includes a first branch lumen and the conduit component further defines a second branch lumen arranged in a Y-shape with the first branch lumen.

9. A method of passing fluids between a patient and a fluid reservoir comprising the steps of:
establishing a fluid flow path between an intraluminal device and a fluid reservoir via a fluid drainage/supply mechanism at least in part via fluidly connecting a proximal tube of the fluid supply/drainage mechanism with the intraluminal device, and fluidly connecting a distal tube of the fluid drainage/supply mechanism with the fluid reservoir;
establishing a twisting force diffusion path between the fluid drainage/supply mechanism and a limb or torso of the patient at least in part via locking the proximal tube to a conduit component of a twist isolation mechanism, locking the distal tube to the conduit component of the twist isolation mechanism, and securing the twist isolation mechanism to the limb or torso of the patient;
passing fluid between the intraluminal device and the fluid reservoir via a fluid lumen defined by the proximal tube, the distal tube, and the conduit component; and
isolating twisting of the proximal tube from twisting of the distal tube during passing the fluid, at least in part by transferring a twisting force according to the twisting force diffusion path from the proximal tube or the distal tube to the limb or torso of the patient.

10. The method of claim 9 further comprising the steps of securing a drainage catheter which includes the intraluminal device to a first part of the patient's body, and securing a collection bag which includes the fluid reservoir to a second part of the patient's body, and wherein the step of passing fluid further includes draining fluid from the drainage catheter to the collection bag.

11. The method of claim 10 further comprising a step of applying a twisting force to one of the proximal tube and the distal tube in response to movement of the patient's body, and wherein the step of isolating further includes the steps of storing a portion of the twisting force within the twist isolation mechanism, and returning the stored portion of the twisting force to the one of the proximal tube and the distal tube in response to another movement of the patient's body.

12. The method of claim 11 wherein the step of storing a portion of the twisting force further includes transmitting the portion of the twisting force to a load reacting outboard edge of a rigid body of the twist isolation mechanism.

13. The method of claim 12 further comprising a step of fluidly disconnecting the distal tube and the fluid collection bag from the conduit component, and connecting an empty fluid collection bag and a substitute distal tube with the conduit component.

14. The method of claim 13 wherein the step of disconnecting includes fluidly disconnecting the collection bag from a first drainage branch lumen defined by the conduit component, and wherein the step of connecting an empty fluid collection bag further includes connecting the empty fluid collection bag to a second drainage branch lumen defined by the conduit component and being in fluid communication with the first drainage branch lumen.

15. A patient-wearable device for isolating twisting of a proximal tube from a distal tube in a fluid drainage/supply system comprising:
a mounting component including a flexible strap having first and second strap ends, and being configured to form an adjustable loop about a limb or torso of a patient;
a control component coupled with the first and second strap ends, and including a rigid body having a proximal body edge, a distal body edge, and first and second load reacting outboard body edges;
a conduit component mounted to the control component and defining a fluid lumen having a proximal lumen end and a distal lumen end;
a proximal coupling component and a distal coupling component connected with the conduit component, and each being configured to lockingly connect with a tube of the fluid drainage/supply system;
the control component further including a patient contact surface having a uniform profile extending from the first outboard body edge to the second outboard body edge, and an upper surface opposite the patient contact surface having a non-uniform profile extending from the first outboard body edge to the second outboard body edge, the non-uniform profile including a raised profile segment defined by the conduit component, and first and second low profile segments defined by the control component; and
the fluid lumen including a segment of a fluid flow path for passing fluids between an intraluminal device and a fluid reservoir, and the patient wearable device defining a twisting force diffusion path between the conduit component and the control component, for limiting transmitting twisting force between the proximal coupling and the distal coupling during passing fluids between the intraluminal device and the fluid reservoir.

16. The device of claim 15 wherein the twisting force diffusion path includes a compound twisting force diffusion path having radial force distributing path segments between the coupling components and the conduit, and tangential force distributing path segments between the conduit component and each of the outboard edges.

17. The device of claim 16 wherein each of the proximal coupling component and the distal coupling component includes a tapered bullet fitting.

18. The device of claim 17 further comprising:
a first and a second buckling post standing proud of the upper surface and each being positioned between one of the load reacting outboard body edges and the conduit component; and
a buckling mechanism having a first buckle coupled with the first buckling post, a second buckle coupled with the second buckling post, and a strap length customizing mechanism engaged with the flexible strap at an adjustable engagement location defining a loop length thereof;
wherein each of the buckles defines a compound aperture positioned about one of the buckling posts, the compound aperture including a minor opening and a major opening, and wherein each of the buckles includes an advanced position at which the corresponding buckling post is positioned within the major opening, and a locked position at which the corresponding buckling post is positioned within the minor opening.

19. The device of claim 15 wherein the fluid lumen includes a first branch lumen and the conduit component further defines a second branch lumen arranged in a Y-shape with the first branch lumen.
